(19) United States
(12) Patent Application Publication
(10) Pub. No.: US 2005/0124554 A1
(43) Pub. Date: Jun. 9, 2005

(54) Spermidine derivatives for the treatment of chronic neurodegenerative diseases

(76) Inventors: Barclay Morrison, London (GB); Ashley Ker Pringle, Hampshire (GB); Lars Eric Sundstrom, Hampshire (GB); Ernst Wulfert, Brussels (BE)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/503,437

(22) PCT Filed: Feb. 3, 2003
(86) PCT No.: PCT/GB03/00507

(30) Foreign Application Priority Data
Feb. 5, 2002 (GB) 0202645.8

(51) Int. Cl. A61K 38/04; A61K 31/16; A61K 31/165

(52) U.S. Cl. 514/19; 514/616; 514/626

(57) ABSTRACT

Compounds of the general formula (I): are useful in treating chronic neurodegenerative diseases or conditions in mammals, such as Alzheimer's Disease, Parkinson's Disease, Huntington's Chorea and Multiple Sclerosis. In formula (I), a=3 or 4; b=3 or 4 provided that a+b≦7; c is an integer from 1 to 5; d is 0 or 1.

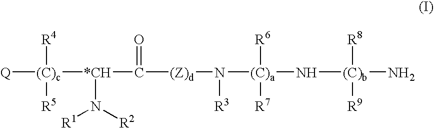

The present invention relates to the treatment of chronic neurodegenerative diseases or conditions, such as Alzheimer's Disease, Parkinson's Disease, Huntington's Chorea and Multiple Sclerosis, as well as to the treatment of other related chronic diseases or disorders, such as chronic inflammatory diseases e.g. rheumatoid arthritis, and inflammatory bowel disease, and other chronic conditions, such as atherosclerosis and other arterial disorders, using neuroprotective compounds. The invention also relates to the protection from or treatment of damage or diseases resulting from superoxide production or peroxynitrite production.

In tissues affected by low oxygen states such as prolonged hypoxia and ischaemia, which may or may not be associated with hypoglycaemia, neuronal damage, to varying degrees, is encountered. Ischaemia typically occurs as a result of an acute event, for example heart attack, stroke or traumatic head injury. During heart attack, the damage incurred is substantially limited to the heart tissues, and certain treatments have been developed. In stroke or traumatic head injury, neuronal damage results from the effects of more long term ischaemia on the brain. The severity of the ischaemia depends on the nature of the stroke or injury, but, invariably, there is brain damage. WO99/31049 addresses the effects of ischaemia on the brain such as occurs with stroke patients or as a result of head injury, and discloses certain neuroprotective agents and their use in treating neuronal damage caused by acute ischaemic events such as stroke and head injury.

In contrast to the neuronal damage occurring as a result of acute ischaemic events such as heart attack, stroke or head injury, the present invention addresses the effects of chronic neurodegenerative diseases or conditions, such as Alzheimer's Disease (AD), Parkinson's Disease (PD), Huntington's Chorea (HC), Multiple Sclerosis (MS) and Amyotrophic Lateral Sclerosis (ALS).

The underlying causes of these neurodegenerative diseases are complex and appear to be multifactorial. In each case, necrotic and apoptotic neuronal cell death may result from one or more mechanisms including metabolic compromise, excitotoxicity and oxidative stress. A number of studies point towards oxidative stress as a major causative factor in a variety of chronic neurodegenerative diseases including AD, PD and ALS (for example, see: Sayre et al., (2001), Curr. Med. Chem, 8(7), 721-38; Bains et al., (1997), Brain Res. Rev., 25, 335-358; Alexi et al. (2000), Progress in Neurobiol., 60, 409-470).

Oxidative stress occurs when the normal balance between oxidative events and antioxidative defence mechanisms is disrupted, either by the loss of reducing agents and/or antioxidants or by increased levels of oxidant species. Oxidative stress has been attributed to the actions of highly toxic free-radicals, including reactive oxide species (ROS) such as the superoxide anion (*O₂⁻) and hydroxyl radical (*OH), and reactive nitrogen species (RNS) derived from nitric oxide (NO) reaction with superoxide or peroxide, such as peroxinitrite (*ONOO⁻).

Excitotoxic cell death is caused by excessive activation of glutamate receptors by glutamate and glutamatergic agonists such as NMDA and other excitatory amino acids (EAAs). A number of studies also suggests that oxidative stress may act as a mediator in excitotoxically induced neuronal cell death. For example, it has been shown for both NMDA and kainate (a non-NMDA receptor agonist) that activation of EAA receptors increases free-radical damage to lipids, and that this damage can be prevented by simultaneous treatment with antioxidants.

Metabolic compromise may be caused by stroke, asphyxiation, hypoglycaemia and certain poisons interfering with mitochondrial respiration. Mitochondrial dysfunction and resulting depletion of ATP and loss of intracellular calcium buffering capacity can cause an increase in the production of reactive oxygen and nitrogen free-radicals, leading to oxidative stress.

Thus, not only is oxidative stress by free-radicals understood to be a primary factor of neuronal cell death in a number of chronic neurodegenerative diseases, but it may also mediate excitotoxic stimuli and metabolic compromise. Furthermore, the reverse interaction may also occur, as oxidative stress by free-radicals may initiate excitotoxic pathways and cause metabolic impairment.

We have now found that certain amine-substituted amide compounds, including compounds disclosed in WO99/31049 for treating of neuronal damage caused by acute ischaemic events such as stroke and head injury, may be used to treat chronic neurodegenerative diseases or conditions in which damage is caused or mediated by free radicals and by excitotoxicity, such as Alzheimer's Disease (AD), Parkinson's Disease (PD), Huntington's Chorea (HC), Multiple Sclerosis (MS) and Amyotrophic Lateral Sclerosis (ALS).

More specifically, we have found, surprisingly, that the compound L-arginyl-3,4-spermidine, previously disclosed in WO99/31049, is neuroprotective against free-radical-induced neuronal cell death in an in vitro model of free-radical-induced injury, and is neuroprotective against EAA mediated cell death in an in vitro model of excitotoxic injury.

Furthermore, we have shown that L-arginyl-3,4-spermidine is neuroprotective against cell death in an in vitro model of ischaemic injury. This in vitro model of ischaemic injury (oxygen and glucose deprivation) is even more severe than the in vitro hypoxia (oxygen deprivation) and in vivo ischaemia models used in WO99/31049. Surprisingly, we have found that L-arginyl-3,4-spermidine is neuroprotective against the much harsher insult of in vitro ischaemic injury not only when administered immediately post injury, but even when administration is delayed up to 60 minutes post injury.

In previous studies, as disclosed in WO99/31049, we have found that administration of L-arginyl-3,4-spermidine did not affect blood pressure, heart rate or respiration, or result in adverse motor function.

In view of the similar structure-activity relationship to L-arginyl-3,4-spermidine of the amine-substituted amide compounds disclosed and tested in the in vitro hypoxia and in vivo ischaemia models according to WO99/131049, it can be expected that the other compounds disclosed and tested in WO99/31049 also exhibit neuroprotective properties against free radical and excitotoxic damage, and against in vitro ischaemic injury. Therefore, in addition to L-arginyl-3,4-spermidine, other compounds also defined by formula (I) or (II) defined below are expected to provide neuroprotection against damage caused or mediated by free radicals and by excitotoxicity, and against in vitro ischaemia, and therefore to be of use in the treatment of chronic neurodegenerative diseases such as AD, PD, HC, MS, and ALS.

Accordingly, in a first aspect, the present invention provides the use of a compound having the general formula (I) for the manufacture of a medicament for treating a chronic neurodegenerative disease or condition: 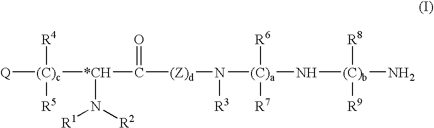
wherein:
Q represents an amidino group, a cyano group or a group of formula XYN—, where
   X and Y are the same or different, and each may represent a hydrogen atom, a lower alkyl group, or a simple hetero-atom containing group or, together with the nitrogen atom to which they are attached, form a nitrogen-containing heterocyclic group;
R¹ and R² are the same as or different from each other and each represents a hydrogen atom, or a group of formula R, RCO—, ROCO—, or RNHCO—, where
   R represents a lower alkyl group or an aryl group, said alkyl or aryl group being optionally substituted by one or more of the substituents α, defined below;
R³ represents a hydrogen atom or a lower alkyl group or an aryl group, said alkyl or aryl group being optionally substituted by one or more of the substituents α, defined below;
R⁴, R⁵, R⁶, R⁷, R⁸ and R⁹ may be the same or different and each represents a hydrogen atom or a lower alkyl group;
the chiral carbon atom indicated by the asterisk is in the L configuration;
Z is an aromatic amino acid residue;
a=3 or 4;
b=3 or 4 provided that a+b≦7;
c is an integer from 1 to 5;
d is 0 or 1;
   and pharmaceutically acceptable salts thereof.

The invention also provides the use of a compound having the general formula (I) and pharmaceutically acceptable salts thereof for the manufacture of a medicament for treating chronic inflammatory diseases e.g. rheumatoid arthritis, and inflammatory bowel disease, and chronic arterial disorders, such as atherosclerosis.

The invention also provides the use of a compound having the general formula (I) and pharmaceutically acceptable salts thereof for the manufacture of a medicament for treating or protecting from damage or diseases resulting from superoxide production.

The invention also provides the use of a compound having the general formula (I) and pharmaceutically acceptable salts thereof for the manufacture of a medicament for treating or protecting from damage or diseases resulting from peroxynitrite production.

Substituents α are selected from: halogen atoms, amino groups, alkylamino groups, dialkylamino groups, cyano groups, hydroxy groups, alkyl groups (except when the substituted group is alkyl), aryl groups, carbamoyl groups, alkylcarbamoyl groups, dialkylcarbamoyl groups and carboxy groups and esters thereof.

In a second aspect, the present invention provides a method of treating a chronic neurodegenerative disease or condition in a mammal comprising administering to said mammal, before or after onset of said disease or condition, an effective amount of a compound as defined above.

Chronic neurodegenerative diseases or conditions that may be treated in accordance with the invention are those in which neuronal cell death or degeneration is mediated by free-radicals, in particular by ROS including the superoxide anion (*O₂⁻), whether caused by oxidative stress, excitotoxic stimulus or metabolic dysfunction, or any combination of these factors. Thus, chronic neurodegenerative diseases or conditions treatable in accordance with the invention include AD, PD, HC, MS and ALS, in particular AD, PD and HC.

Without wishing to be bound to a particular mechanism or theory, it is believed that the compounds used in accordance with the invention function as biological scavengers, by inactivating, consuming, quenching, neutralising or reducing free radicals and ROS.

The compounds used according to the present invention are preferably prepared in substantially pure form. By substantially pure is meant a compound which, under conditions of HPLC (high performance liquid chromatography) is not shown to have any or any significant amount of contaminants detectable thereby. Trace levels of contaminants may be acceptable in certain circumstances and such circumstances may be determined by the skilled person at the time. In general, levels of contaminant should be less than 1%, and preferably substantially less than 1%, for example less than 0.1%, possibly as low as 0.001%. In the alternative, it is preferred that the compounds are non-toxic, by which is meant that the compounds should not exhibit any unacceptable levels of toxicity at the dosages at which they are applied. Preferably, they should exhibit no toxicity whatsoever.

Regardless of the foregoing, the class of compounds defined above is useful in treating chronic neurodegenerative diseases or conditions, based on our tests on the hippocampus, as described below.

The neuroprotective compounds herein described may be administered to patients as a prophylactic in order to prevent onset of a chronic neurodegenerative disease. In addition or alternatively, the compounds may be applied after onset of a chronic neurodegenerative disease, but it will be appreciated that it is preferred to administer the compounds as soon as possible, in order to avoid as much neuronal degeneration as possible. In most circumstances it will be desirable to administer repeated doses at least whilst the patient continues to show symptoms of the disease.

Suitable methods of administration are generally by injection, in order to achieve the desired result as soon as possible, but are not limited to this route. Thus, intravenous injection is particularly preferred but, in some circumstances it may be preferable to administer the compound directly into the cerebrospinal fluid.

The dose of the compound of the present invention will vary depending upon many factors, including the age, body weight and general condition of the patient, as well as the mode, frequency and route of administration. However, a dose of from 0.01 to 50 mg/kg body weight is generally recommended, a dose of from 0.05 to 20 mg/kg body weight being more preferred. This may be administered in a single dose or in divided doses, more preferably in a course of periodic, for example daily, weekly or monthly doses.

Preferably, the compound is used or administered in an amount effective to provide a dosage in the range of 0.3 to 300 microM, more preferably in the range of 3 to 300 microM.

In the compounds of the present invention, it is generally preferred that the overall length of the compound is in the region of the length of compound (Ia), as shown hereafter. Compound (Ia) can be considered to be 18 units long, so that we prefer the compounds of the present invention should be no longer than 25, more preferably no longer than 22, units long, and no shorter than 14 units long. This is a general preference, but it is generally noted that there is a rapid drop-off in activity with a length change of any significance, even one unit having a generally undesirable effect. Accordingly, it is more preferred that the compound should be from 17 to 22, more preferably from 16 to 22, units long. By “unit” is meant an atom in the longest chain, excluding hydrogen, and those non-chain atoms attached thereto. Thus, for example, in formula (I), the group —NH₂ is regarded as a unit, as are the groups —NH—, —CH(NR¹R²)—, —C(O)—, etc.

As mentioned, Q may represent an amidino group, a cyano group or a group of formula XYN—.

Where X or Y represents a lower alkyl group, this has from 1 to 6 carbon atoms and may be a straight or branched chain group having from 1 to 6, preferably from 1 to 4, carbon atoms. Examples include the methyl, ethyl, propyl, isopropyl, butyl, isobutyl, sec-butyl, t-butyl, pentyl, isopentyl, neopentyl, 2-methylbutyl, 1-ethylpropyl, 4-methylpentyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, hexyl and isohexyl groups. Of these, we prefer those alkyl groups having from 1 to 4 carbon atoms, preferably the methyl, ethyl, propyl, isopropyl, butyl and isobutyl groups, and most preferably the methyl group.

Where X or Y represents a simple hetero-atom containing group, this may be an acyclic or cyclic group. Examples of acyclic groups include the amidino group (to form, with the nitrogen atom to which X and Y are attached, a guanidino group), alkoxycarbonyl groups (to form an alkoxycarbonylamino group), the carbamoyl group or thiocarbamoyl group (to form the ureido group or the thioureido group). Examples of heterocyclic groups which may be represented by X and Y include those groups having from 5 to 10 ring atoms (in one or two rings), of which from 1 to 4 are nitrogen and/or oxygen and/or sulphur hetero-atoms, the remainder being carbon atoms. Where there are 4 hetero-atoms, we prefer that all 4 are nitrogen atoms. Where there are 3 hetero-atoms, we prefer that all 3, 2 or 1 are nitrogen atoms. Where there are 2 hetero-atoms, we prefer that 2 or 1 are nitrogen atoms. Examples of such groups include the pyrrolyl, tetrazolyl, indolyl, thiazolyl, furyl, pyranyl, chromenyl, imidazolyl, pyrazolyl, isothiazolyl, oxazolyl, isoxazolyl, pyridyl, pyrazinyl, pyrimidinyl, isoindolyl, quinolyl, isoquinolyl, carbazolyl, chromanyl, pyrrolidinyl, pyrrolinyl, imidazolidinyl, piperidyl, piperazinyl, indolinyl and morpholinyl groups.

Alternatively, X and Y, together with the nitrogen atom to which they are attached, may form a nitrogen-containing heterocyclic group. Examples of such heterocyclic groups include those groups having from 5 to 10 ring atoms (in one or two rings), of which from 1 to 4 are nitrogen and/or oxygen and/or sulphur hetero-atoms, the remainder being carbon atoms. Where there are 4 hetero-atoms, we prefer that all 4 are nitrogen atoms. Where there are 3 hetero-atoms, we prefer that all 3, 2 or 1 are nitrogen atoms. Where there are 2 hetero-atoms, we prefer that 2 or 1 are nitrogen atoms. Examples of such groups include the 1-pyrrolyl, 1- or 2-tetrazolyl, 1-indolyl, 3-thiazolyl, 1-imidazolyl, 1-pyrazolyl, 2-isothiazolyl, 3-oxazolyl, 2-isoxazolyl, 1-pyridyl, 1-pyrazinyl, 1-isoindolyl, 1-quinolyl, 2-isoquinolyl, 9-carbazolyl, 1-pyrrolidinyl, 1-pyrrolinyl, 1-imidazolidinyl, piperidino, 1-piperazinyl, 1-indolinyl and morpholino groups.

Where Q represents an alkoxycarbonylamino group, the alkoxy part preferably has from 1 to 6 carbon atoms and may be a straight or branched chain group. Examples of such groups include the methoxycarbonylamino, ethoxycarbonylamino, propoxycarbonylamino, isopropoxycarbonylamino, butoxycarbonylamino, pentyloxycarbonylamino and hexyloxycarbonylamino groups, of which we prefer those groups having from 1 to 4 carbon atoms, and most prefer the ethoxycarbonylamino group.

Preferably at least one of X and Y represents a hydrogen atom. We particularly prefer that one or both of X and Y represents a hydrogen atom. Particularly preferred compounds are those compounds of formula (I) in which both X and Y represent hydrogen atoms or those in which one of X and Y represents a hydrogen atom and the other represents an amidino group or a carbamoyl group. The most preferred compounds are those compounds of formula (1) in which both X and Y represent hydrogen atoms or those in which one of X and Y represents a hydrogen atom and the other represents an amidino group.

The various groups R, R³, R⁴, R⁵, R⁶ R⁷, R⁸ and R⁹ may be lower alkyl or aryl groups which may be unsubstituted or may be substituted by at least one of substituents α, defined above. The lower alkyl groups have from 1 to 6 carbon atoms, and examples include the methyl, ethyl, propyl, isopropyl, butyl, isobutyl, sec-butyl, t-butyl, pentyl, isopentyl, neopentyl, hexyl and isohexyl groups, of which the methyl and ethyl groups are preferred, the methyl group being most preferred. The aryl groups are carbocyclic aromatic groups which preferably have from 6 to 10 ring carbon atoms, and more preferably have 6 or 10 ring carbon atoms, for example the phenyl, 1-naphthyl and 2-naphthyl groups, of which the phenyl group is preferred. Alternatively, any of these groups may be substituted by one or more of substituents α.

Examples of substituents α include:

halogen atoms for example chlorine, fluorine or bromine atoms;
amino groups;
alkylamino groups, in which the alkyl part preferably has from 1 to 6 carbon atoms, for example the methylamino, ethylamino, propylamino, butylamino, t-butylamino, pentylamino and hexylamino groups;
dialkylamino groups, in which the alkyl part preferably has from 1 to 6 carbon atoms, for example the dimethylamino, diethylamino, methylethylamino, dipropylamino, dibutylamino, dipentylamino and dihexylamino groups;
cyano groups;
hydroxy groups;
alkyl groups (except when the substituted group is alkyl), for example as exemplified above in relation to R, R³ etc.;
aryl groups, for example as exemplified above in relation to R, R³ etc.;
carbamoyl groups;
alkylcarbamoyl groups, in which the alkyl part preferably has from 1 to 6 carbon atoms, for example the methylcarbamoyl, ethylcarbamoyl, propylcarbamoyl, butylcarbamoyl, t-butylcarbamoyl, pentylcarbamoyl and hexylcarbamoyl groups; and
dialkylcarbamoyl groups, in which the alkyl part preferably has from 1 to 6 carbon atoms, for example the dimethylcarbamoyl, diethylcarbamoyl, methylethylcarbamoyl, dipropylcarbamoyl, dibutylcarbamoyl, dipentylcarbamoyl and dihexylcarbamoyl groups.

Examples of such substituted groups include: halogen-substituted methyl groups, preferably having three halogen atoms, such as the trichloromethyl and trifluoromethyl groups; halogen-substituted phenyl groups, such as the o-, m- and p-chlorophenyl, o-, m- and p-fluorophenyl, o-, m- and p-bromophenyl, 2,3-dichlorophenyl, 2,3-difluorophenyl, 3,4-dichlorophenyl, 3,4-difluorophenyl, 2,4,6-trichlorophenyl and 2,4,6-trifluorophenyl groups; amino-substituted alkyl groups, such as the aminomethyl, 2-aminoethyl, 3-aminopropyl and 4-aminobutyl groups; alkylamino-substituted alkyl groups (in which the alkyl part of the alkylamino group preferably has from 1 to 4 carbon atoms), such as the methylaminomethyl, 2-methylaminoethyl, 3-methylaminopropyl, 4-methylaminobutyl, ethylaminomethyl, 2-ethylaminoethyl, 3-ethylaminopropyl, 4-ethylaminobutyl, propylaminomethyl, 2-propylaminoethyl, 3-propylaminopropyl, 4-propylaminobutyl, butylaminomethyl, 2-butylaminoethyl, 3-butylaminopropyl and 4-butylaminobutyl groups; dialkylamino-substituted alkyl groups (in which each alkyl part of the dialkylamino group preferably has from 1 to 4 carbon atoms), such as the N,N-dimethylaminomethyl, 2-N,N-dimethylaminoethyl, 3-N,N-dimethylaminopropyl, 4-N,N-dimethylaminobutyl, N,N-diethylaminomethyl, 2-N,N-diethylaminoethyl, 3-N,N-diethylaminopropyl, 4-N,N-ethylaminobutyl, N,N-propylaminomethyl, 2-N,N-propylaminoethyl, 3-N,N-propylaminopropyl, 4-N,N-propylaminobutyl, N,N-butylaminomethyl, 2-N,N-butylaminoethyl, 3-N,N-butylaminopropyl and 4N,N-butylaminobutyl groups; aryl- (particularly phenyl or naphthyl) substituted alkyl groups, such as the benzyl, phenethyl, 3-phenylpropyl or 4-phenylbutyl groups; carbamoyl-substituted alkyl groups, such as the carbamoylmethyl, 2-carbamoylethyl, 3-carbamoylpropyl and 4-carbamoylbutyl groups; alkylcarbamoyl-substituted alkyl groups (in which the alkyl part of the alkylcarbamoyl group preferably has from 1 to 4 carbon atoms), such as the methylcarbamoylmethyl, 2-methylcarbamoylethyl, 3-methylcarbamoylpropyl, 4-methylcarbamoylbutyl, ethylcarbamoylmethyl, 2-ethylcarbamoylethyl, 3-ethylcarbamoylpropyl, 4-ethyl-carbamoylbutyl, propylcarbamoylmethyl, 2-propylcarbamoylethyl, 3-propylcarbamoylpropyl, 4-propylcarbamoylbutyl, butylcarbamoylmethyl, 2-butylcarbamoylethyl, 3-butylcarbamnoylpropyl and 4-butylcarbamoylbutyl groups; dialkylcarbamoyl-substituted alkyl groups (in which each alkyl part of the dialkylcarbamoyl group preferably has from 1 to 4 carbon atoms), such as the N,N-dimethylcarbamoylmethyl, 2-N,N-dimethylcarbamoylethyl, 3-N,N-dimethylcarbamoylpropyl, 4-N,N-dimethylcarbamoylbutyl, N,N-diethylcarbamoylmethyl, 2-N,N-diethylcarbamoylethyl, 3-N,N-diethylcarbamoylpropyl, 4-N,N-ethylcarbamoylbutyl, N,N-propylcarbamoylmethyl, 2-N,N-propylcarbamoylethyl, 3-N,N-propylcarbamoylpropyl, 4-N,N-propylcarbamoylbutyl, N,N-butylcarbamoylmethyl, 2-N,N-butylcarbamoylethyl, 3-N,N-butylcarbamoylpropyl and 4-N,N-butylcarbamoylbutyl groups; carboxy-substituted alkyl groups, such as the carboxymethyl, 2-carboxyethyl, 3-carboxypropyl and 4-carboxybutyl groups and esters thereof, and o-, m- and p-aminophenyl, methylaminophenyl, ethylaminophenyl, propylaminophenyl, butylaminophenyl, N,N-dimethylaminophenyl, N,N-diethylaminophenyl, N,N-dipropylaminophenyl, N,N-dibutylaminophenyl, biphenylyl, carbamoylphenyl, methylcarbamoylphenyl, ethylcarbamoylphenyl, propylcarbamoylphenyl, butylcarbamoylphenyl, N,N-dimethylcarbamoylphenyl, N,N-diethylcarbamoylphenyl, N,N-dipropylcarbamoylphenyl, N,N-dibutylcarbamoylphenyl and carboxyphenyl groups and esters of the carboxyphenyl groups.

Examples of ester groups include:
alkyl groups having from 1 to 20 carbon atoms, more preferably from 1 to 6 carbon atoms, such as those exemplified above and higher alkyl groups as are well known in the art, such as the heptyl, octyl, nonyl, decyl, dodecyl, tridecyl, pentadecyl, octadecyl, nonadecyl and icosyl groups, but most preferably the methyl, ethyl and t-butyl groups;
cycloalkyl groups having from 3 to 7 carbon atoms, for example the cyclopropyl, cyclobutyl, cyclopentyl, cyclohexyl and cycloheptyl groups;
aralkyl groups, in which the alkyl part has from 1 to 3 carbon atoms and the aryl part is a carbocyclic aromatic group having from 6 to 14 carbon atoms, which may be substituted or unsubstituted and, if substituted, has at least one of substituents a defined and exemplified above, although the unsubstituted groups are preferred; examples of such aralkyl groups include the benzyl, phenethyl, 1-phenylethyl, 3-phenylpropyl, 2-phenylpropyl, 1-naphthylmethyl, 2-naphthylmethyl, 2-(1-naphthyl)ethyl, 2-(2-naphthyl)ethyl, benzhydryl (i.e. diphenylmethyl), triphenylmethyl, bis(o-nitrophenyl)-methyl, 9-anthrylmethyl, 2,4,6-trimethylbenzyl, 4-bromobenzyl, 2-nitrobenzyl, 4-nitrobenzyl 3-nitrobenzyl, 4-methoxybenzyl and piperonyl groups;
alkenyl groups having from 2 to 6 carbon atoms, such as the vinyl, allyl, 2-methylallyl, 1-propenyl, isopropenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl and 5-hexenyl groups, of which the vinyl, allyl, 2-methylallyl, 1-propenyl, isopropenyl and butenyl groups are preferred, the allyl and 2-methylallyl groups being most preferred.
halogenated alkyl groups having from 1 to 6, preferably from 1 to 4, carbon atoms, in which the alkyl part is as defined and exemplified in relation to the alkyl groups above, and the halogen atom is chlorine, fluorine, bromine or iodine, such as the 2,2,2-trichloroethyl, 2-halo ethyl (e.g. 2-chloro ethyl, 2-fluoroethyl, 2-bromo ethyl or 2-iodoethyl), 2,2-dibromoethyl and 2,2,2-tribromoethyl groups;
substituted silylalkyl groups, in which the alkyl part is as defined and exemplified above, and the silyl group has up to 3 substituents selected from alkyl groups having from 1 to 6 carbon atoms and phenyl groups which are unsubstituted or have at least one substituent selected from substituents a defined and exemplified above, for example a 2-trimethylsilylethyl group;
phenyl groups, in which the phenyl group is unsubstituted or substituted, preferably with at least one alkyl group having from 1 to 4 carbon atoms or acylamino group, for example the phenyl, tolyl and benzamidophenyl groups;
phenacyl groups, which may be unsubstituted or have at least one of substituents α defined and exemplified above, for example the phenacyl group itself or the p-bromo-phenacyl group;
cyclic and acyclic terpenyl groups, for example the geranyl, neryl, linalyl, phytyl, menthyl (especially m- and p-menthyl), thujyl, caryl, pinanyl, bornyl, notcaryl, norpinanyl, norbornyl, menthenyl, camphenyl and norbornenyl groups;
alkoxymethyl groups, in which the alkoxy part has from 1 to 6, preferably from 1 to 4, carbon atoms and may itself be substituted by a single unsubstituted alkoxy group, such as the methoxymethyl, ethoxymethyl, propoxymethyl, isopropoxymethyl, butoxymethyl and methoxyethoxymethyl groups;
aliphatic acyloxyalkyl groups, in which the acyl group is preferably an alkanoyl group and is more preferably an alkanoyl group having from 2 to 6 carbon atoms, and the alkyl part has from 1 to 6, and preferably from 1 to 4, carbon atoms such as the acetoxymethyl, propionyloxymethyl, butyryloxymethyl, isobutyryloxymethyl, pivaloyloxymethyl, 1-pivaloyloxyethyl, 1-acetoxyethyl, 1-isobutyryloxyethyl, 1-pivaloyloxypropyl, 2-methyl-1-pivaloyloxypropyl, 2-pivaloyloxypropyl, 1-isobutyryloxyethyl 1-isobutloxypropyl, 1-acetoxypropyl, 1-acetoxy-2-methylpropyl, 1-propionyloxyethyl, 1-propionyloxypropyl, 2-acetoxypropyl and 1-butyryloxyethyl groups;
cycloalkyl-substituted aliphatic acyloxyalkyl groups, in which the acyl group is preferably an alkanoyl group and is more preferably an alkanoyl group having from 2 to 6 carbon atoms, the cycloalkyl substituent has from 3 to 7 carbon atoms, and the allyl part has from 1 to 6, preferably from 1 to 4, carbon atoms, such as the (cyclohexyl acetoxy)methyl, 1-(cyclohexylacetoxy)ethyl, 1-(cyclohexylacetoxy)propyl, 2-methyl-1-(cyclohexylacetoxy)propyl, (cyclopentylacetoxy)methyl, 1-(cyclopentylacetoxy)ethyl, 1-(cyclopentylacetoxy)propyl and 2-methyl-1-(cyclopentylacetoxy)propyl groups;
alkoxycarbonyloxyalkyl groups, especially 1-(alkoxycarbonyloxy)ethyl groups, in which the alkoxy part has from 1 to 10, preferably from 1 to 6, and more preferably from 1 to 4, carbon atoms, and the alkyl part has from 1 to 6, preferably from 1 to 4, carbon atoms, such as the 1-methoxycarbonyloxyethyl, 1-ethoxycarbonyloxyethyl, I-propoxycarbonyloxyethyl, 1-isopropoxycarbonyloxyethyl, 1-butoxycarbonyloxyethyl, 1-isobutoxycarbonyloxyethyl, 1-sec-butoxycarbonyloxyethyl, 1-t-butoxycarbonyloxyethyl, 1-(1-ethylpropoxycarbonyloxy)ethyl and 1-(1,1-dipropylbutoxycarbonyloxy)ethyl groups, and other alkoxycarbonylalkyl groups, in which both the alkoxy and alkyd groups have from 1 to 6, preferably from 1 to 4, carbon atoms, such as the 2-methyl-1-(isopropoxycarbonyloxy)propyl, 2-(isopropoxycarbonyloxy)propyl, isopropoxycarbonyloxymethyl, t-butoxycarbonyloxymethyl, methoxycarbonyloxymethyl and ethoxycarbonyloxymethyl groups;
cycloalkylcarbonyloxyalkyl and cycloalkyloxycarbonyloxyalkyl groups, in which the cycloalkyl group has from 3 to 10, preferably from 3 to 7, carbon atoms, is mono- or poly-cyclic and is optionally substituted by at least one (and preferably only one) alkyl group having from 1 to 4 carbon atoms (e.g. selected from those alkyl groups exemplified above) and the alkyl part has from 1 to 6, more preferably from 1 to 4, carbon atoms (e.g. selected from those alkyl groups exemplified above) and is most preferably methyl, ethyl or propyl, for example the 1-methylcyclohexylcarbonyloxymethyl, 1-methylcyclohexyloxycarbonyloxymethyl, cyclopentyloxycarbonyloxymethyl, cyclopentylcarbonyloxymethyl, 1-cyclohexyloxycarbonyloxyethyl, 1-cyclohexylcarbonyloxyethyl, 1-cyclopentyloxycarbonyloxyethyl, 1-cyclopentylcarbonyloxyethyl, 1-cycloheptyloxycarbonyloxyethyl, 1-cycloheptylcarbonyloxyethyl, 1-methylcyclopentylcarbonyloxymethyl, 1-methylcyclopentyloxycarbonyloxymethyl, 2-methyl-1-(1-methylcyclohexylcarbonyloxy)propyl, 1-(1-methylcyclohexylcarbonyloxy)propyl, 2-(1-methylcyclohexylcarbonyloxy)propyl, 1-(cyclohexylcarbonyloxy)propyl, 2-(cyclohexylcarbonyloxy)propyl, 2-methyl-1-(1-methylcyclopentylcarbonyloxy)propyl, 1-(1-methylcyclopentylcarbonyloxy)propyl, 2-(1-methylcyclopentylcarbonyloxy)propyl, 1-(cyclopentylcarbonyloxy)propyl, 2-(cyclopentylcarbonyloxy)propyl, 1-(1-methylcyclopentylcarbonyloxy)ethyl, 1-(1-methylcyclopentylcarbonyloxy)propyl, adamantyloxycarbonyloxymethyl, adamantylcarbonyloxymethyl, 1-adarnantyloxycarbonyloxyethyl and 1-adamantylcarbonyloxyethyl groups;
cycloalkylalkoxycarbonyloxyalkyl groups in which the alkoxy group has a single cycloalkyl substituent, the cycloalkyl substituent having from 3 to 10, preferably from 3 to 7, carbon atoms and mono- or poly-cyclic, for example the cyclopropylmethoxycarbonyloxymethyl, cyclobutylmethoxycarbonyloxymethyl, cyclopentylmethoxycarbonyloxymethyl, cyclohexylmethoxycarbonyloxymethyl, 1-(cyclopropylmethoxycarbonyloxy)ethyl, 1-(cyclobutylmethoxycarbonyloxy)ethyl, 1-(cyclopentylmethoxycarbonyloxy)ethyl and 1-(cyclohexylmethoxycarbonyloxy)ethyl groups;
terpenylcarbonyloxyalkyl and terpenyloxycarbonyloxyalkyl groups, in which the terpenyl group is as exemplified above, and is preferably a cyclic terpenyl group, for example the 1-(menthyloxycarbonyloxy)ethyl, 1-(menthylcarbonyloxy)ethyl, menthyloxycarbonyloxymethyl, menthylcarbonyloxymethyl, 1-(3-pinanyloxycarbonyloxy)ethyl, 1-(3-pinanylcarbonyloxy)ethyl, 3-pinanyloxycarbonyloxymethyl and 3-pinanylcarbonyloxymethyl groups;
5-alkyl or 5-phenyl [which may be substituted by at least one of substituents α, defined and exemplified above] (2-oxo-1,3-dioxolen-4-yl)alkyl groups in which each alkyl group (which may be the same or different) has from 1 to 6, preferably from 1 to 4, carbon atoms, for example the (5-methyl-2-oxo-1,3-dioxolen-4-yl)methyl, (5-phenyl-2-oxo-1,3-dioxolen-4-yl)methyl, (5-isopropyl-2-oxo-1,3-dioxolen-4-yl)-methyl, (5-t-butyl-2-oxo-1,3-dioxolen-4-yl)methyl and 1-(5-methyl-2-oxo-1,3-dioxolen-4-yl)ethyl groups; and
other groups, especially groups which are easily removed in vivo such as the phthalidyl, indanyl and 2-oxo-4,5,6,7-tetrahydro-1,3-benzodioxolen-4-yl groups.

Of the above groups, we especially prefer those groups which can be removed easily in vivo, and most preferably the aliphatic acyloxyalkyl groups, alkoxycarbonyloxyalkyl groups, cycloalkylcarbonyloxyalkyl groups, phthalidyl groups and (5-substituted 2-oxo-1,3-dioxolen-4-yl)methyl groups.

However, we prefer that R, R³, R⁴, R⁵, R⁶, R⁷, R⁸, and R⁹ are all hydrogen.

It is generally preferred that the group Z is not present, i.e. d is 0, but where it is present then it corresponds to the residue of an aromatic amino acid (i.e. an amino acid, preferably an α- or β-amino acid, containing one or more aromatic groups), preferably a hydrophobic aromatic amino acid. Preferred are aromatic α-amino acids, such as histidine, phenylalanine, tyrosine, tryptophan or phenylglycine, of which phenylalanine or tyrosine are most preferred.

In a preferred embodiment of the first aspect and second aspects, the compound used has the general formula (II): 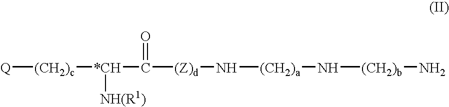
wherein:
Q represents a group selected from NH₂C(NH)NH—, NH₂C(O)NH—, NH₂—, NH₂C(O)— or imidazol-4-yl;
the chiral carbon atom indicated by the asterisk is in the L configuration;
Z is an aromatic α-amino acid residue —NH—CH(R¹⁰)—CO— oriented left to right in formula (II), in which the α-amino acid is selected from phenylalanine, tyrosine, tryptophan and phenylglycine whereby R¹⁰ correspondingly represents CH₂Ph, —CH₂PhOH, CH₂-3-indole or —Ph;
R¹ represents a hydrogen atom or a group selected from —CH₂Ph, —C(O)OCH₂Ph and —C(O)CH₃;
a=3 or 4;
b=3 or 4 provided that a+b≦7;
c is an integer from 1 to 4;
d=0 or 1;
   and pharmaceutically acceptable salts thereof.

Preferred compounds of general formula (I) or formula (II) are those in which a+b=7, and in particular where a=3 and b=4. Preferably c=3 or 4. Preferably d=0 whereby no group Z is present. Preferably, Q represents a group selected from NH₂C(NH)NH— and NH₂C(O)NH—. Preferably, R¹ represents a hydrogen atom or a group selected from —CH₂Ph and —C(O)OCH₂Ph (where Ph phenyl).

Especially preferred are the following specific compounds:

### Compound (Ia) L-arginyl-3,4-spermidine (“L-Arg3,4”):

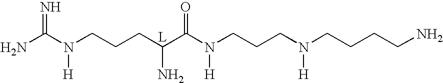

### Compound (Ib) L-lysinyl-3,4-spermidine (“Lys3,4”):

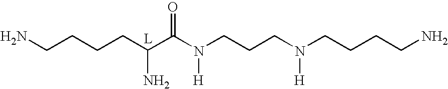

### Compound (Ic) L-ornithinyl-3,4-spermidine (“Orn3,4”):

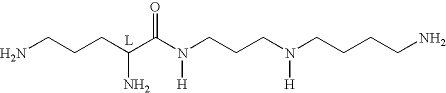

### Compound (Id) (“HomoArgPhe-3,4”):

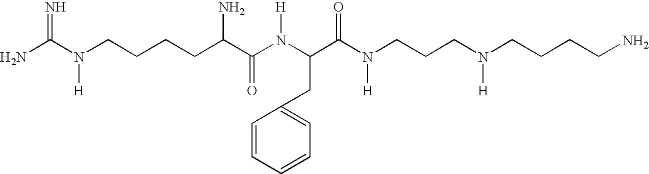

### Compound (Ie) (“ArgTyr3,4”):

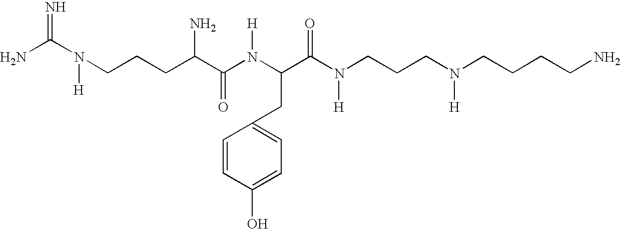

### Compound (If) (“ArgTrp3,4”):

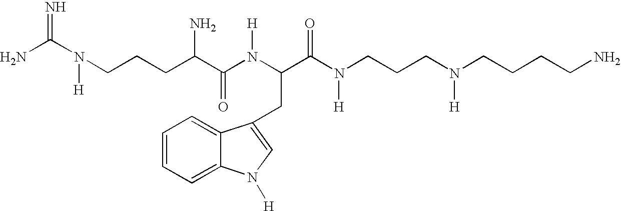

### Compound (Ig) L-arginyl-L-phenylalaninyl-3,4-spermidine (“ArgPhe3,4”):

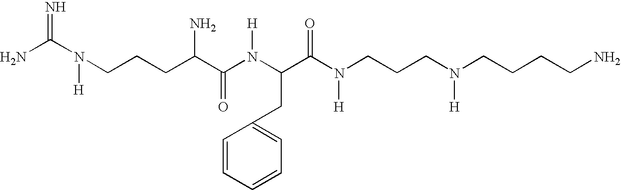

### Compound (Ih) (“ArPhg3,4”):

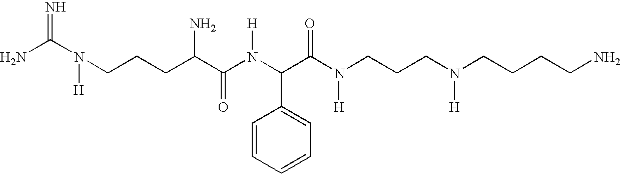

### Compound (Ii) L-citrullinyl-3,4-spermidine (“Cit3,4”):

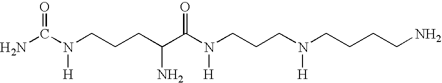

Of these, the compounds of formula (Ia), (Id), (Ie), (If), (Ig), (Ih) and (Ii) are especially preferred, the compounds of formula (Ia) and (Id) being more preferred, and the compound of formula (Ia) being most preferred.

The compounds of the present invention may be prepared by a variety of processes which, in themselves, are well-known in the art, for example as disclosed in WO99/31049.

Preparation of an exemplary compound of the invention, as well as neuroprotective activity is illustrated in the accompanying non-limiting examples:

### EXAMPLE 1

Materials

Drugs: Kainate, NMDA, glutamate, AMPA, and 6,7-Dinitroquinoxaline-2,3(1H,4H)-dione (DNQX) were purchased from Sigma (UK). DNQX was stored at room temperature in DMSO at 40 mM. All other drugs were made up as aqueous solutions.

Chemicals and Reagents: Propidium iodide (PI) was purchased form Molecular Probes (Eugene, Oreg.). Dimethyl sulfoxide (DMSO), glucose and glutamine were purchased from Sigma. Millicell CM culture inserts were purchased from Millipore (UK). The 1N-Dde-8N-Mmt-spermidine-4-yl)-carbonyl Wang polystyrene resin was purchased from Novabiochem (Switzerland), and the Fmoc-Arg(Boc)₂-OH from Bayer (UK). All other chemicals and reagents were obtained from commercial sources except for L-arginyl-3,4-spermidine (“L-Arg3,4”) which was synthesised as indicated below.

Compound Synthesis

L-Arg3,4 Trifluoroacetic Acid Salt was synthesised as follows:

(1N−1-(4,4-dimethyl-2,6-dioxocyclohexylidene)ethyl-8N-monomethoxytrityl-spermidine-4-yl)-carbonyl Wang polystyrene resin (0.279 g, loading 0.42 mmol gag 0.12 mmol) was swollen in N,N-dimethylformamide (DMF). The solvent was then drained and 2% hydrazine in DMF (4 ml) was added. The resulting mixture was spun for 5 min. The solution was drained and the hydrazinolysis repeated twice more. The resin was then washed with DMF (×3), DCM (×3), and diethyl ether (×3). A qualitative ninhydrin test was positive.

The resin was then swollen in DMF and the solvent drained. A solution of 9-fluorenylmethoxycarbonyl-arginine-(tert-butyloxycarbonyl)₂-OH (0.142 g, 0.24 mmol, 0.24 M) and N-hydroxybenzotriazole hydrate (0.037 g, 0.24 mmol, 0.24 M) in dichloromethane (DCM) (0.6 ml) and DMF (0.4 ml) were stirred at room temperature for 10 min, diisopropylcarbodiimide (38 μl, 0.24 mmol) was added and the resulting solution stirred for a further 10 min. The reaction mixture was then added to the damp resin and spun for 2.5 h. The solution was drained and the resin washed with DMW (×3), DCM (×3) and diethyl ether (×3). A qualitative ninhydrin test was negative.

The resin was swollen in DMF and the solvent drained. A 20% piperidine solution in DMF (4 ml) was added and the reaction mixture was spun for 10 min. The solution was drained and the piperidine treatment repeated. The resin was washed with DMF (×3), DCM (×3) and diethyl ether (×3). A quantitative ninhydrin test was positive.

To the dry resin was added a solution of 97.5% trifluoroacetic acid (TEA) and 2.5% triisopropylsilane (4 ml), and the reaction mixture allowed to stand for 3 h. The solvent was then drained and the resin washed with TFA (2×1 ml). The combined acid phases were evaporated in vacuo. The residue was dissolved in TFA (0.4 ml) and precipitated in ice-cold diethyl ether. The mixture was centrifuged and the solvent removed by decanting. The pellet was washed with diethyl ether, the mixture centrifuged, and the solvent again decanted. The residue was then dried in vacuo overnight. The residue was dissolved in TFA (0.4 ml) and the diethyl ether precipitation repeated. The sample was then dissolved in water and acetonitrile and lyophilised. This was repeated once more to afford L-Arg3,4 as a sticky orange-brown solid (0.061 g, 20% L-Arg3,4 by mass). The compound was quantified by ¹H nuclear magnetic resonance spectroscopy with phenol as an internal standard.

### EXAMPLE 2

Experiments were conducted using L-Arg3,4 as synthesised according to Example 1:

(i) Protocol for Preparation of Organotypic Hippocampal Slice Cultures:

Organotypic hippocampal slice cultures were prepared according to established methods (Pringle et al. (1997), Brain Res., 755, 36-46). In brief, Wistar rat pups (8-11 days old) were decapitated and the hippocampus rapidly dissected into ice-cold Gey's balanced salt solution (Gibco Life Technologies, Paisley, UK) supplemented with 4.5 mg ml⁻¹ glucose. Slices were separated and plated onto Millicell CM culture inserts (4 per well) and maintained at 37° C. and 5% CO₂ for 14 days. Maintenance medium consisted of 25% heat-inactivated horse serum, 25% Hank's balanced salt solution (HBSS) and 50% minimum essential medium with added Earle's salts MM, ICN Biomedicals, Basingstoke, UK) supplemented with 1 mM glutamine and 4.5 mg ml⁻¹ glucose. Medium was changed every 3-4 days.

(ii) Protocol for Studying Effect of Ischaemic Injury:

Experimental ischaemia or oxygen glucose deprivation was performed as described previously (Pringle et al. (1996), Stroke, 27, 2124-2130). Briefly, cultures were transferred to serum-free media (SFM, 75% MEM, 25% HDSS supplemented with 1 mM glutamine and 4.5 mg ml⁻¹ glucose) containing 5 μg ml⁻¹ of the fluorescent exclusion dye propidium iodide (PI). Cultures were allowed to equilibrate in SFM for 30 min prior to imaging, with or without L-Arg3,4. PI fluorescence was detected using a Leica inverted microscope as described below. Any cultures in which PI fluorescence was detected at this stage were excluded from further study. Ischaemia was induced by transferring cultures to glucose-free media (MEM with Earl's salts without glucose and supplemented with 1 mM glutamine) saturated with 95% N₂ and 5% CO₂. Culture plates (without lids) were then sealed into an airtight chamber (Billups-Rothenberg, Del Mar, Calif.) gassed with 95% N₂ and 5% CO₂ at 10 L min⁻¹ for 10 min before being sealed and placed at 37° C. for 50 min. At the end of the ischaemia, cultures were returned to normoxic SFM+PI, with or without compound at the indicated concentrations, and placed back in the incubator for 24 h.

(iii) Protocol for Studying Effect of Excitotoxic Injury:

Excitotoxic injury was performed as previously described (Pringle et al. (1997), Brain Res., 755, 36-46) with L-Arg3,4 present pre-, during, and post-injury fin treated cultures. Cultures were transferred to SFM+PI with or without 300 μM L-Arg3,4 and allowed to equilibrate for 30 min prior to imaging. Any cultures exhibiting PI fluorescence were excluded. The medium was then exchanged for SFM+PI plus a glutamate receptor agonist (either 10 mM glutamate, 10 μM NMDA, 10 μM AMPA, or 10 μM kainate) for 3 h. After exposure, the media was again exchanged for SFM+PI containing 300 μM L-Arg3,4. Cultures were then returned to the incubator until they were assessed for cell death in the indicated region of the pyramidal cell layer 24 h post exposure.

(iv) Protocol for Studying Effect of Free-Radical Injury:

Free-radical mediated injury was performed as described previously with L-Arg3,4 present pre-, during, and post-injury in treated cultures (Wilde et al. (1997, J. Neurochem., 69, 883-886). Briefly, cultures were transferred to SFM+PI with or without 300 μM L-Arg3,4 and allowed to equilibrate for 30 min prior to imaging. Any cultures in which PI fluorescence was detected at this stage were excluded from further study. Duroquinone (DQ) was dissolved in DMSO at a stock concentration of 100 mM which was then diluted 1:1000 in SFM for a final working concentration of 100 μM (final DMSO concentration of 0.1%). Free-radical injury was initiated by transferring cultures to SFM supplemented with 100 μM duroquinone for 3 h. At the end of the exposure, cultures were returned to SFM+PI, with or without compound, and placed in the incubator for 24 h.

(v) Protocol for Assessment of Cell Death:

Neuronal damage was assessed as described previously (Pringle et al. (1996), Stroke, 27, 2124-2130; Pringle et al. (1997), Brain Res., 755, 36-46). Light transmission images were captured prior to the induction of injury, and PI fluorescence images recorded at the end of the 24 h post-injury recovery period. All images were captured on an inverted Leica DM-IRBE epifluorescence microscope (Milton Keynes, UK) fitted with a 100 W Hg lamp and standard rhodamine optics (excitation 510-560 nm; dichroic mirror 620 nm; emission>590 nm). Images were acquired with a 5×NA 0.12 lens and a cooled Hamamatsu digital camera and digitised at 12 bit resolution for analysis in OpenLab 2.1 (Improvision, Coventry, UK) running on a Macintosh G4/400. The area of the CA1 or CA3 cell layer was determined from the transmission image, and the area of PI fluorescence in that cell layers was measured using the density slice function within OpenLab. Neuronal damage was expressed as a percentage of the area in which PI fluorescence was detected above threshold within a cell layer divided by the total area of that cell layer. Protection was calculated as the difference between the damage for a given well and the average damage in untreated controls divided by the average damage in untreated controls. Groups were compared against corresponding control groups using the two tailed Student's t-test with significance indicated as * for P<0.05, and ** for P<0.01. Data is presented as mean±s.e.mean.

(vi) Protocol for Studying Electrophysiology:

All animal procedures to produce acute hippocampal slices were approved by the Home Office. Male, 250 g, Sprague-Dawley rats were deeply anaesthetised with halothane and decapitated. The brain was quickly removed, and the hippocampi dissected out and then cut into 300 μm thick transverse slices on a vibratome (Leica, Milton Keynes, UK) in cold cutting solution (in mM: 189 sucrose, 2.5 KCl, 26 NaHCO₃, 1.2 NaH₂PO₄, 10 glucose, 5 MgCl₂, 0.1 CaCl₂) equilibrated with 5% CO₂, 95% O₂. Slices were transferred to aCSF (in mM: 10 glucose, 125 NaCl, 5 KCl, 26 NaHCO₃, 1 MgCl₂, 1.25 NaH₂PO₄, 2 CaCl₂, 10 sucrose) at room temperature, bubbled with 5% CO₂ and 95% O₂, for at least 1 h before use.

Slices were then transferred to a submerged perfusion chamber (2 ml volume) for the electrophysiological recordings and were perfused at 10 ml min⁻¹ at 25° C. with aCSF equilibrated with 5% CO₂ and 95% O₂, and allowed to equilibrate for 1 h before manipulations were started. Experimental compounds were applied by bath perfusion. L-Arg3,4 was applied at 300 μM and DNQX was applied at 10 μM final concentration. DNQX was diluted from a 40 mM stock in DMSO (final DMSO of 0.025%). The Schaffer collaterals of CA3 were stimulated at voltages from 0 to 30 V with a constant voltage stimulator (Digitimer, Hertfordshire, UK) via a twisted pair stimulating electrode of polyimide coated stainless steel wire (Plastics One, Roanoke, Va.). Field potential responses were recorded with an Axopatch 200B amplifier (Axon Instruments, Foster City, Calif.) in the fast current clamp mode via a 2M KCl filled glass electrode (2-5 MQ) in the CA1 pyramidal cell layer. Responses were filtered at 2 kHz, digitised at 20 kHz with a Digidata 1320A (Axon Instruments), and stored onto hard disk with Clampex 8.1 software (Axon Instruments) running on a Dell Pentium III PC. Data was analysed in Clampfit 8.1 (Axon Instruments).

Stimulus-response (S/R) curves were generated from stimuli from 0 to 30 V in 2 V increments. Bath perfusion of 300 μM L-Arg3,4 in aCSF was applied for 10 min with a sub-maximal stimulus applied every minute to monitor synaptic transmission. A second S/R curve was generated before the perfusion was switched back to plain aCSF. A sub-maximal stimulus was applied every minute during the 15 min wash period after which a third SIR curve was generated. Perfusion of 10 μM DNQX was then started until the response to the sub-maximal stimulus was completely eliminated which always occurred within 10 min. Increasing the stimulus to maximum (30V) did not generate a response, and therefore, no S/R curves were generated in the presence of DNQX. In Clampfit, S/R curves were fit to a sigmoidal function of the form:

$$R = \frac{{R}_{Max}}{1 + {ⅇ}^{m \cdot ( {V}_{50} - S )}}$$

where R is the magnitude of the evoked response, Rₘₐₓ is the maximum response, V₅₀ is the voltage which produces a half maximal response, S is the stimulus voltage, and m is proportional to the slope of the linear region of the sigmoid. Data was compared by one way ANOVA.

Hydroethidine (HEt) has been utilised to fluorescently monitor the production of superoxide in primary neuronal cultures [Bindokas et al., (1996), Carriedo et al. (1998)]. A stock solution of HEt was made was made in N₂ sparged DMSO (10 mg ml⁻¹) which was then aliquoted and stored at −80° C. aliquots were used only once before being discarded. Two separate experimental paradigms were employed to test the effect of L-Arg3,4 on superoxide production in response to either AMPA or NMDA stimulation.

For the AMA experiments, organotypic slice cultures were loaded with 1 μg ml⁻¹ HEt in HEPES buffered salt solution (HSS) (in mM): glucose 10, NaCl 144, KCl 5, HEPES 10, MgCl₂ 1, CaCl₂ 2, pH 7.4, for 30 minutes in the dark. All subsequent solutions contained HEt to prevent a decrease in signal due to reporter depletion. Images were acquired once per minute for the duration of the experiment on the same system as described above for assessment of cell death. A 10 minute record of baseline superoxide production was produced before the bathing solution was exchanged for HBSS+BEt containing 10 μM AMPA, again for 10 minutes. As shown previously, stimulation of glutamate receptors with AMPA lead to an increase in superoxide production [Bindokas et al., (1996), Carriedo et al. (1998)]. The bathing solution was then exchanged for HBSS+HEt containing both 10 μM AMPA and 300 μM L-Arg3,4 for 10 minutes to test the effect of L-Arg3,4 on superoxide production. In separate control experiments, 300 μM L-Arg3,4 in HSS was incubated with slices to determine if it affected baseline superoxide production on its own.

Acquired images from the AMPA experiments were then analysed with the OpenLab image analysis package. A region of interest was drawn around the CA1 pyramidal cell layer because this was the cell layer that produced the largest increase in fluorescence in response to AMPA activation. In each image, within the region of interest, the average pixel value was calculated in arbitrary units (AU) to produce a record of fluorescence intensity values over time. A linear regression was then performed on the data to calculate the rate of superoxide production as change in intensity over time (arbitrary units per minute, AU min⁻¹) for each of the treatments. Data is presented as mean±s.d. Rates of superoxide production were then compared by ANOVA followed by post-hoc comparisons with Bonferroni corrections with significance indicated as * for P<0.05 and ** for P<0.01.

Results (Example 2):

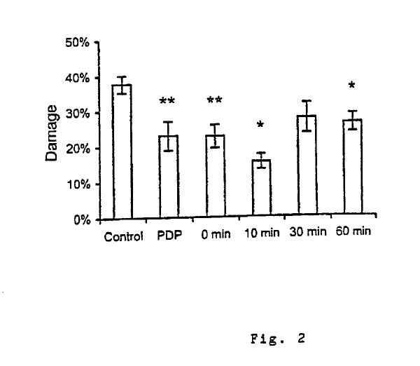

The results are depicted in FIGS. 2 to 7.

(i) Effect of L-Arm3.4 on Ischaemic Cell Death:

One hour of ischaemia generated reproducible neurodegeneration in the pyramidal cell layer in hippocampal slice cultures of approximately 38±2% as determined by PI staining. Cell death in untreated control cultures from individual ischaemia experiments was not statistically different (P=0.70), and, therefore, ischaemic controls were combined into a single group (FIG. 2: “Control”) (n=113). Incubation of cultures in 300 μM L-Arg3,4 pre-, during, and post-ischaemia resulted in a significant reduction of neurodegeneration (FIG. 2: “PDP”) to 23±4% (39% protection, P<0.01, n=34).

(ii) Effect of L-Arg3.4 on Ischaemic Cell Death when Administration is Delayed:

To determine the effective time window after injury, administration of L-Arg3,4 was delayed until after the completion of the ischaemia and applied at 0, 10, 30, and 60 min post-injury. Delayed application of 300 μM L-Arg3,4 significantly decreased cell death as compared to controls at 0, 10, and 60 min post-injury (FIG. 2) reducing damage to 23±3% (40% protection, P<0.01, n=36), 15±2% (59% protection, P<0.01, n=24), and 26±3% (30% protection, P<0.05, n=31), respectively. Although damage was reduced to 28±4% at 30 min post injury, the 26% protection did not reach statistical significance (P<0.08, n=25).

(iii) Effect of Varying Dose of L-Arg3.4 on Ischaemic Cell Death:

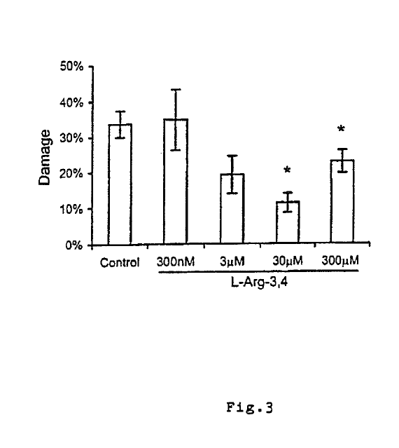

The effective post-injury dose for neuroprotection against ischaemia was tested with concentrations of L-Arg3,4 ranging from 300 nM to 300 μM. A dose of 300 μM was initially chosen for all studies based on the data above in a model of hypoxic injury which indicated that 300 μM was an optimal dose. Untreated controls from separate experiments were not significantly different (P=0.9) with an aggregate damage of 34±9% and were combined into a single group (FIG. 3: “Control”) (n=47). At 300 nM, L-Arg3,4 did not reduce damage, however, at 3, 30, and 300 μM, the compound did reduce damage to 19±5% (43% protection, P<0.06, n=14), 11±3% (67% protection, P<0.05, n=8), and 23±3% (32% protection, P<0.05, n=36), respectively as compared to the untreated group (FIG. 3).

(iv) Effect of L-Arg3,4 on Excitotoxic Cell Death:

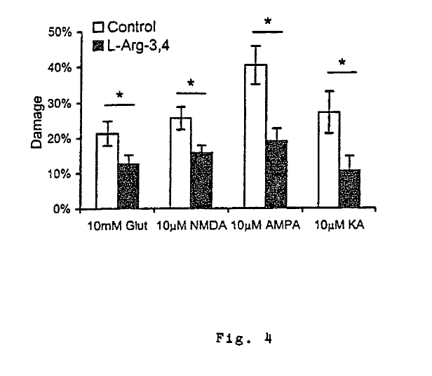

Administration of 300 μM L-Arg3,4 significantly reduced cell death in the CA1 cell layer generated by 10 mM Glut (FIG. 4) from 21±3% damage in controls (n=23) to 13±3% in treated cultures (41% protection, P<0.05, n=23) suggesting that it could be a glutamate receptor antagonist. To determine if its actions were glutamate receptor subtype specific, L-Arg3,4 was tested against 10 μM NMDA, 10 μM AMPA, and 10 μM kainate in separate experiments which produced regionally specific cell death. Administration of 300 μM L-Arg3,4 significantly reduced the cell death in CA1 generated by 10 μM NMDA (FIG. 4) from 25±3% damage in controls (n=42) to 16±2% in treated cultures (38% protection, P<0.05, n=43) or 10 μM AMPA (FIG. 4) from 41±5% damage in controls (1=21) to 19±4% in treated cultures (53% protection, P<0.01, n=22). Furthermore, 300 μM L-Arg3,4 significantly reduced the cell death in CA3 due to 10 μM kainate (FIG. 4: “KA”) from 27±6% damage in controls (n=21) to 11±4% in treated cultures (61% protection, P<0.05, I=24).

(v) Effect of L-Arg3.4 on Synaptic Transmission:

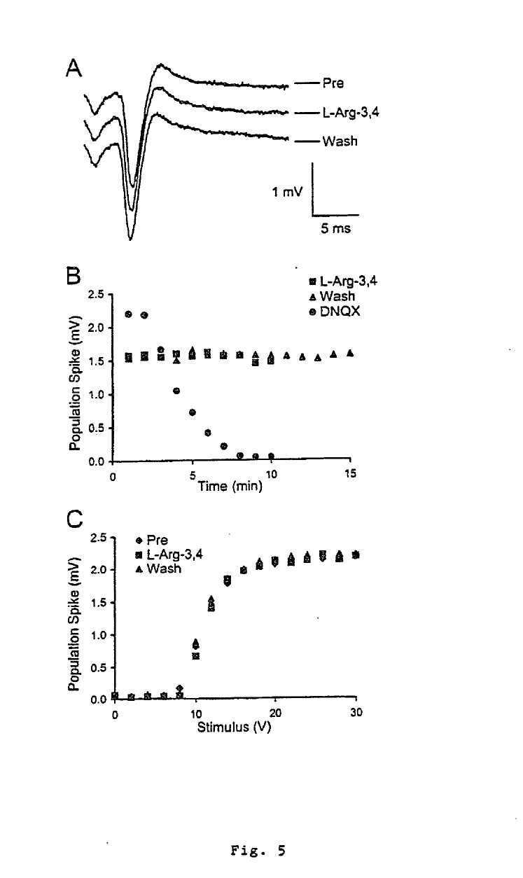

Bath application of 300 μM L-Arg3,4 did not grossly affect evoked responses from acute hippocampal slices. Individual evoked responses from a single slice before, during, and after L-Arg3,4 application showed that the compound had no effect on evoked responses (FIG. 5A). This lack of effect was in contrast to the effect of 10 μM DNQX which blocked the evoked response completely (FIG. 5B). The population spike of a single slice to a sub-maximal stimulus was not affected by L-Arg3,4 infusion nor its wash out. However, in the same slice, 10 μM DNQX eliminated all transmission within 5 minutes (FIG. 5B). S/R curves for a slice before, during, and after L-Arg3,4 were not affected by bath application of 300 μM L-Arg3,4 (FIG. 5C). All S/R curves were fitted to a sigmoidal function so that comparisons between aggregate SIR curves could be made (Table 1). None of the parameters was significantly affected by L-Arg3,4 infusion as determined by a one way ANOVA for each parameter.

TABLE 1

| | Rₘₐₓ (mV) | V₅₀ (V) | m | n |
|---|---|---|---|---|
| Pre | 2.61 ± 1.16 | 19.28 ± 6.02 | 0.32 ± 0.18 | 6 |
| Arg-3,4 | 2.71 ± 1.31 | 15.98 ± 5.12 | 0.59 ± 0.48 | 6 |
| Wash | 2.38 ± 0.83 | 15.07 ± 5.39 | 0.78 ± 0.60 | 3 |
| P | 0.92 | 0.48 | 0.29 | |

(vi) Effect of L-Arg3.4 on Free-Radical Mediated Cell Death:

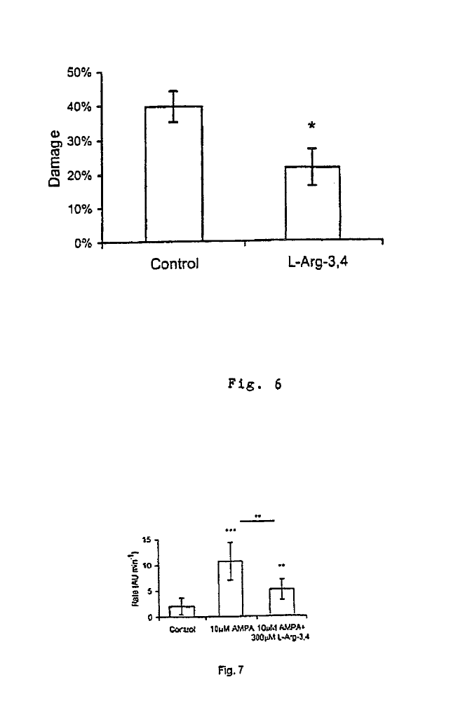

Administration of 300 μM L-Arg3,4 significantly reduced cell death in the CA1 pyramidal cell layer caused by a 3 h incubation in 100 μM duroquinone (FIG. 6), indicating that it is quenching free-radicals. Injury was reduced from 40±5% in untreated controls (71=21) to 21±5% in treated cultures protection 46%, P<0.05, n=21).

In addition to blocking free-radical mediated damage, we have also demonstrated that L-Arg3,4 significantly reduces the production of superoxide radicals by AMPA implying that the compound has a direct effect on free-radical production.

(vii) Effect of L-Arg3,4 on Superoxide Production)

Production of superoxide was reliably measured fluorescently with HEt as described by Bindokas et al. (1996) and Carriedo et al. (1998). In the AMPA stimulation experiments, baseline superoxide production was measured to be 2.06±1.60 AU min⁻¹ (n=16) and was not affected by 300 μM L-Arg3,4, 2.55±2.02 AU min⁻¹ (P>0.6, data not shown). Stimulation of glutamate receptors by 10 μM AMPA significantly increased the rate of superoxide production approximately 5 fold to 10.65±3.70 AU min-1 (n=18, P<0.001, FIG. 7). The subsequent addition of 300 μM L-Arg3,4, in the continued presence of 10 μM AMPA, significantly reduced the rate of superoxide production by approximately half to 5.07±1.94 AU min-d compared to the rate with AMPA alone (n=4, P<0.01), although this rate was still significantly elevated from baseline (P<0.01).

Discussion of Results (Example 2):

Organotypic hippocampal slice culture models are ideal preparations for the search of novel drugs because they allow for the rapid screening of a large number of compounds. These culture preparations possess the added advantage over other in vitro systems of maintaining several key similarities to in vivo models including delayed neurodegeneration and selective vulnerability of either the CA1 or CA3 pyramidal cell layer depending on the injury paradigm and severity.

We have extended previous results obtained with a model of hypoxia to a more severe in vitro injury paradigm of ischaemia (oxygen glucose deprivation) and have shown that L-Arg3,4 significantly prevented CA1 cell loss after 1 h of ischaemia (FIG. 2). To determine the available therapeutic window for L-Arg3,4, administration was delayed for up to 60 min post-ischaemia and, surprisingly, it was still neuroprotective. This therapeutic window is over twice as long as that for MK-801, D-APV, or dextromethorphan which lose effectiveness if administration is delayed for 30 min in models of excitotoxicity (Hartley & Choi, (1989), J Pharmacol. Exp. Ther., 250, 752-758), suggesting that the mechanism of action of L-Arg3,4 is unlikely to be at the level of the NMDA receptor.

Given the well established involvement of glutamate in the pathophysiological sequelae of ischaemia, we tested whether L-Arg3,4 was neuroprotective against EAA mediated cell death. L-Arg3,4 significantly prevented cell death due to glutamate, NMDA, AMPA, and kainate toxicity.

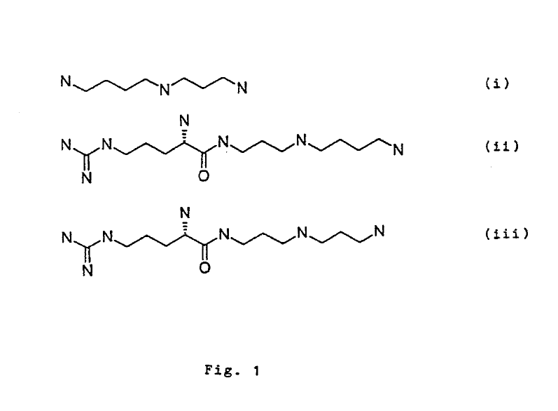

Given its structural similarity to spermidine (FIG. 1(i)), L-Arg3,4 could be acting at the polyamine site of the NMDA receptor. L-Arg3,4 (FIG. 1(ii)) does not potentiate NMDA receptor activation because it did not exacerbate NMDA mediated neurotoxicity in our studies, and therefore, probably does not interact with the polyamine site on the NMDA receptor.

In our experiments, L-Arg3,4 did not affect synaptic transmission in any way as determined by measuring evoked field responses in acute hippocampal slices (FIG. 5) reaffirming that it does not interact with voltage sensitive calcium channels (VSCC). This lack of effect was in sharp contrast to the effect of 10 μM DNQX which abolished all synaptic transmission within 10 min of exposure indicating that L-Arg3,4 does not alter transmission mediated by non-NMDA receptors.

Its lack of electrophysiological effects in combination with its ability to prevent ischaemic and excitotoxic cell death by both NMDA and non-NMDA receptor agonists suggests that L-Arg3,4 acts down stream of receptor and or channel activation. Its very long therapeutic window also suggests that it may target a nexus of the pathological sequelae which is common to these models of cell death especially since NMDA antagonists have a much shorter therapeutic window of less than 30 min in models of excitotoxicity (Hartley & Choi, (1989), J Pharmacol. Exp. Ther. 250, 752-758).

To test the hypothesis that the protection afforded by L-Arg3,4 was due to its ability to react with ROS, cultures were injured by incubation in 100 μM duroquinone which specifically generates superoxide by disruption of the mitochondria electron transport chain (Wilde et al., (1997). J. Neurochem., 69, 883-886). Surprisingly, L-Arg3,4 significantly protected against free-radical mediated injury suggesting that it can quench ROS (FIG. 6).

In summary, we have studied the neuroprotective mechanism of action of L-Arg3,4, which prevents cell death in an in vitro ischaemia model. Surprisingly, delayed administration of L-Arg3,4 after ischaemia (up to 60 min) was also neuroprotective. This extremely long therapeutic window in vitro is in contrast to NMDA antagonists, such as MK-801, which lose efficacy as early as 15 min post-injury (Hartley & Choi, (1989) J. Pharmacol. Exp. Ther., 250, 752-758). The compound prevented cell death due to glutamate but was not receptor sub-type specific as it also prevented cell death due to NMDA, AMPA, and kainate. The compound did not inhibit synaptic transmission, and therefore, was not likely to directly antagonise glutamate receptors or VSCC unlike a structurally similar compound, sFTX-3.3 (FIG. 1 (iii)). L-Arg3,4 also prevented superoxide mediated cell death. Because it did not affect synaptic transmission, L-Arg3,4 may engender fewer detrimental side effects than previous, experimental, therapeutic compounds.

1. The use of a compound having the general formula (I) for the manufacture of a medicament for treating a chronic neurodegenerative disease or condition: 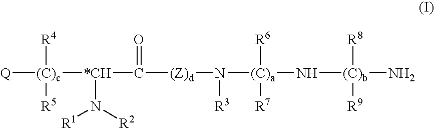
wherein:
Q represents an amidino group, a cyano group or a group of formula XYN—, where
X and Y are the same or different, and each may represent a hydrogen atom, a lower alkyl group, or a simple hetero-atom containing group or, together with the nitrogen atom to which they are attached, form a nitrogen-containing heterocyclic group;
R¹ and R² are the same as or different from each other and each represents a hydrogen atom, or a group of formula R, RCO—, ROCO—, or RNHCO—, where
R represents a lower alkyl group or an aryl group, said alkyl or aryl group being optionally substituted by one or more of the substituents α, defined below;
R³ represents a hydrogen atom or a lower alkyl group or an aryl group, said alkyl or aryl group being optionally substituted by one or more of the substituents α, defined below;
R⁴, R⁵, R⁶, R⁷, R⁸ and R⁹ may be the same or different and each represents a hydrogen atom or a lower alkyl group;
the chiral carbon atom indicated by the asterisk is in the L configuration;
Z is an aromatic amino acid residue;
substituents α are selected from: halogen atoms, amino groups, alkylamino groups, dialkylamino groups, cyano groups, hydroxy groups, alkyl groups (except when the substituted group is alkyl), aryl groups, carbamoyl groups, alkylcarbamoyl groups, dialkylcarbamoyl groups and carboxy groups and esters thereof;
a=3 or 4;
b=3 or 4 provided that a+b≦7;
c is an integer from 1 to 5;
d is 0 or 1;
and pharmaceutically acceptable salts thereof.

2. The use according to claim 1 wherein the chronic neurodegenerative disease or condition is selected from Alzheimer's Disease, Parkinson's Disease, Huntington's Chorea, Multiple Sclerosis and Amyotrophic Lateral Sclerosis.

3. The use according to claim 1 or claim 2, wherein the compound is of the general formula (II): 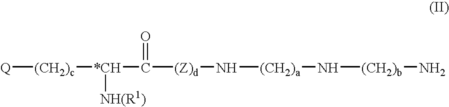
wherein:
Q represents a group selected from NH₂C(NH)NH—, NH₂C(O)NH—, NH₂—, NH₂C(O)— or imidazol-4-yl;
the chiral carbon atom indicated by the asterisk is in the L configuration;
Z is an aromatic α-amino acid residue —NH—CH(R¹⁰)—CO— oriented left to right in formula (II), in which the α-amino acid is selected from phenylalanine, tyrosine, tryptophan and phenylglycine whereby R¹⁰ correspondingly represents CH₂Ph, —H₂PhOH, CH₂-3-indole or —Ph;
R¹ represents a hydrogen atom or a group selected from —CH₂Ph, —C(O)OCH₂Ph and —C(O)CH₃;
a=3 or 4;
b=3 or 4 provided that a+b≦7;
c is an integer from 1 to 4;
d=0 or 1;
and pharmaceutically acceptable salts thereof.

4. The use according to any preceding claim wherein a+b=7.

5. The use according to claim 4 wherein a 3 and b=4.

6. The use according to any preceding claim wherein c=3 or 4

7. The use according to any preceding claim wherein d=0.

8. The use according to any preceding claim wherein Q represents a group selected from NH₂C(NH)NH— and NH₂C(O)NH—.

9. The use according to any preceding claim wherein R¹ represents a hydrogen atom or a group selected from —CH₂Ph and —C(O)OCH₂Ph.

10. The use according to claim 1 or claim 2, wherein the compound is:

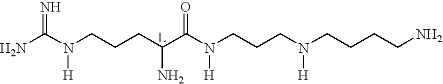

11. The use according to claim 1 or claim 2, wherein the compound is:

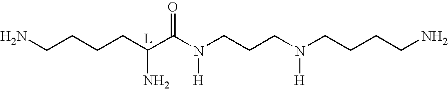

12. The use according to claim 1 or claim 2, wherein the compound is:

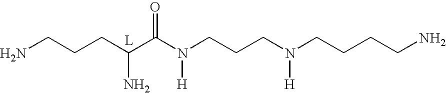

13. The use according to claim 1 or claim 2, wherein the compound is:

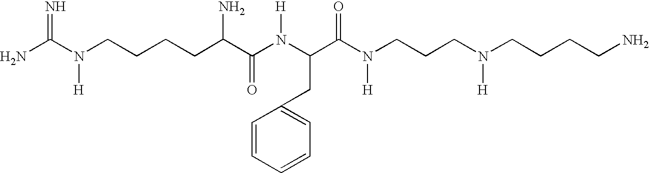

14. The use according to claim 1 or claim 2, wherein the compound is:

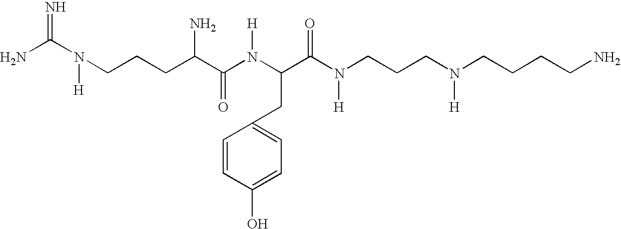

15. The use according to claim 1 or claim 2, wherein the compound is:

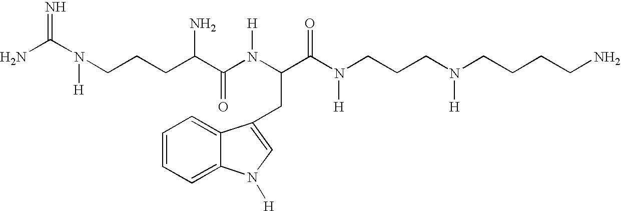

16. The use according to claim 1 or claim 2, wherein the compound is:

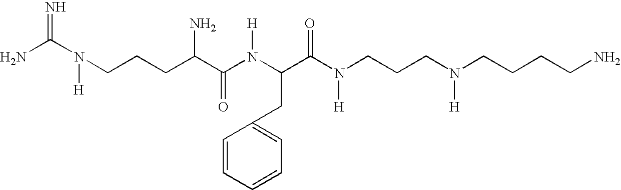

17. The use according to claim 1 or claim 2, wherein the compound is:

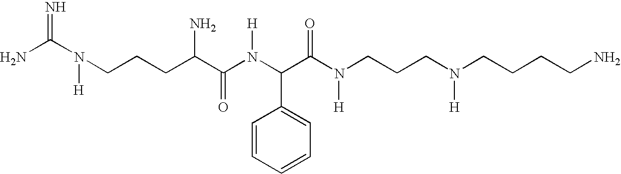

18. The use according to claim 1 or claim 2, wherein the compound is:

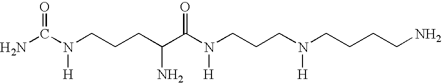

19. A method of treating a chronic neurodegenerative disease or condition in a mammal comprising administering to said mammal, before or after onset of said disease or condition, an effective amount of a compound as defined in any one of claims 1 to 18.

20. The use or method according to any preceding claim wherein the compound is used or administered in an amount effective to provide a dosage in the range of 0.3 to 300 μM.

21. The use or method according to claim 19 wherein the compound is used or administered in an amount effective to provide a dosage in the range of 3 to 300 μM.

22. The use of a compound according to any one of claims 1 to 18 for the manufacture of a medicament for treating chronic inflammatory diseases, such as rheumatoid arthritis and inflammatory bowel disease, and chronic arterial disorders, such as atherosclerosis.

23. The use of a compound according to any one of claims 1 to 18 for the manufacture of a medicament for treating or protecting from damage or diseases resulting from superoxide production.

24. The use of a compound according to any one of claims 1 to 18 for the manufacture of a medicament for treating or protecting from damage or diseases resulting from peroxynitrite production.

25. A method of treating chronic inflammatory diseases, such as rheumatoid arthritis and inflammatory bowel disease, and chronic arterial disorders, such as atherosclerosis in a mammal comprising administering to said mammal, before or after onset of said disease or disorder, an effective amount of a compound as defined in any one of claims 1 to 18.

26. A method of treating or protecting from damage or diseases resulting from superoxide production in a mammal comprising administering to said mammal, before or after onset of said disease or disorder, an effective amount of a compound as defined in any one of claims 1 to 18.

27. A method of treating or protecting from damage or diseases resulting from peroxynitrite production in a mammal comprising administering to said mammal, before or after onset of said disease or disorder, an effective amount of a compound as defined in any one of claims 1 to 18.

28. The use or method according to claim 23 or claim 26, wherein said compound has the formula: [chemical structure]
